(19) United States
(12) Patent Application Publication
(10) Pub. No.: US 2011/0304820 A1
(43) Pub. Date: Dec. 15, 2011

(54) METHOD AND DEVICE FOR IMAGING A TARGET

(75) Inventors: Pauli Falt, Joensuu (FI); Jouni Hiltunen, Joensuu (FI)

(73) Assignee: KUOMED OY, Tampere (FI)

(21) Appl. No.: 13/133,744

(22) PCT Filed: Dec. 10, 2009
(86) PCT No.: PCT/FI2009/050996

§ 371 Date: Aug. 29, 2011

(30) Foreign Application Priority Data
Dec. 12, 2008 (FI) 20086189

(51) Int. Cl. A61B 3/14

(52) U.S. Cl. 351/206; 351/246

(57) ABSTRACT

The present invention relates to a method and apparatus for forming a filtered image filtered with an at least partially negative filtering spectrum. In the method a plurality of exposure combinations of exposure times and wavelength channels are determined so that at least some of the wavelength channels are on the positive area of the filtering spectrum, on which area the exposure is essentially determined to correspond with the filtering spectrum, while at least some of the wavelength channels are on the negative area of the filtering spectrum, on which area the exposure is determined to essentially correspond with the inverted filtering spectrum. Partial images are formed of the target using the said combinations of exposure times and wavelength channels by illuminating the target with the light source, by filtering the light emitted by the source of light and by detecting the light reflected or transmitted. Finally, the said image filtered with a partially negative filtering spectrum is formed of the partial images by calculation.

### FIELD OF THE INVENTION

The present invention relates to forming an image of a target filtered by means of a desired filtering spectrum. The invention especially relates to carrying out a partially negative filtering for imaging targets of various types. An application area of the invention is medical imaging, especially imaging the ocular fundus and the epidermis.

### BACKGROUND OF THE INVENTION

Spectral imaging of a target differs from traditional digital photography in that instead of the RGB data (R=red, G=green, B=blue) each pixel contains data of the light spectrum directed to the detector element of the whole image detector corresponding to the pixel. As the whole spectrum cannot be saved at one go with traditional image detectors, in order to produce a spectral image a plurality of images must be made of the target at different wavelength bands. Compared to traditional digital images the spectral images contain multiple amounts of memory space.

In a number of research methods based on digital imaging of the target to be studied in the field of e.g. medical practices it is often desirable to filter the light illuminating the target with an exactly defined filter spectrum. Thus it would be possible to visualize in the target features that cannot be detected by other means. However, producing a filter capable of producing the desired filtering spectrum as such can be very difficult or even impossible.

Various research groups have developed methods based on various techniques for producing filters having desired spectrum forms either by means of physical filters or by means of digital post-processing. Filtering pictures has been widely researched especially in medical applications in which it is necessary to combine or interpret the information produced by means of a number of methods and different conditions.

U.S. Pat. No. 6,788,822 discloses a method in which the brightness of a digital image can be corrected so that the dark areas of the image can be made brighter and vice versa. The method is based on the Multiscale Retinex process using the retina of the eye as its model.

Published patent application US 2003/053668 relates to combining two images so that one or more grayscale areas can be shown from the grayscale histogram of the images for forming a combined image. The application of the method is, e.g. combining images taken of the same target by means of a number of medical apparatuses.

U.S. Pat. No. 5,672,877 discloses a method in which information of images taken from the same target by means of a number of methods are coregistered.

Published patent application US 2007/092124 shows a method by means of which it is possible to form a combination of two images so that in the field of view corresponding to a certain target area the combined image is formed as a subtraction of two images, whereby the changes occurring over time in the target area are made visible in the combined image while showing also the parts surrounding the area of the target of the imaged target.

Published patent application US 2006/013500 publishes a method and apparatus for Super Montage Large Area Spectroscopic Imaging. The principles of the method include forming large image areas as montages of a number of partial images with different wavelengths, compensating imaging mistakes at each wavelength and combining the partial images of each wavelength for forming a spectroscopic image.

The method and apparatus published in published patent application US 2008/081029 relate to resolving a certain target area of a digital image. In the solution the target is illuminated both through a filter comprising a certain spatial frequency and without a filter. The subtraction of the image is transformed into a spatial frequency space whereby also the spatial frequency properties of each pixel are found out. The spatial frequency properties of each pixel are compared to the corresponding properties of the filter.

Published patent application US 2003/215153 discloses a method for processing an image saved on a medium having non-linear response to light for producing a digital image with improved contrast. In the method, e.g. local filter and hue scale are used for further modifying the original and the subtraction images formed in different ways.

U.S. Pat. No. 5,717,605 shows a system that can be utilized for forming images filtered with negative filtering spectra. The system, however, utilizes integrating light detection at various wavelength bands, whereby weak detection dynamic forms a problem. Further, wavelength band-specific exposure time is adjusted on the exposure side, which further decreases the signal to noise ratio of imaging.

U.S. Pat. No. 5,379,065 illustrates a further imaging method which is, however, not suitable for carrying out partially negative filtering spectra.

U.S. Pat. No. 6,504,943 discloses a filtering solution in which the filtering of the wavelength channels is carried out only after the target in the detector side. This increases the amount of light in the target and limits the use of the method in e.g. medical imaging applications due to low patient safety.

Jääskeläinen et al (T. Jaaskelainen, S. Toyooka, S. Izawa and H. Kadono, Color classification by vector subspace method and its optical implementation using liquid crystal spatial light modulator, Optics Communications 89, 23,-29 (1992)) disclose a so-called vector subspace method for displaying colour data and for classifying colours. They have used diffraction grids and a spatial light modulator for producing truly positive filters.

Fauch et al (L. Fauch, E. Nippolainen, A. A. Kamshilin, M. Hauta-Kasari, J. Parkkinen and T. Jääskeläinen, Optical Implementation of Precise Color Classification Using Computer Controlled Set of Light Emitting Diodes, Optical review 14(4), 243-245 (2007)) describe a programmable light source based on LED light sources, adjustable acoustic-optical filter by means of which the spectrum of the target can be formed with the so-called principal component analysis, PCA. The method utilizes an integrating light detector summing temporally consecutively the wavelength bands produced by the LED light sources and the filter. The disadvantage of the apparatus in practical solutions is that the light power produced therewith is very low and two-dimensional imaging would require very long exposure times. The integrating light detector is also very easily saturated as the light intensity increases. Thus the method and apparatus are not usable for, e.g. medical imaging.

Styles et al (Styles, I. B., Calcagni, A., Claridge, E., Orihuele-Espina, F., Gibson, J. M.: Quantitative analysis of multi-spectral fundus images, Med. Image Anal. 10, 578-597 (2006))—have disclosed a method for spectral imaging of the ocular fundus. In the method, a number of images are made of the ocular fundus with different wavelengths using liquid crystal filters. Johnson et al. (Johnson, W. R., Wilson, D. W., Fink, W., Humayun, M., Bearman, G.: Snapshot hyperspectral imaging in ophtalmology. J. Biomed. Opt. 12, 014036 (2007)) disclose as a variation of this method using a diffractive optical element, by means of which the wavelengths of white light are locally separated before the measurement of their intensity. The calculations necessary for the method are relatively intensive.

In practice, none of the above-mentioned publications disclose a practically effective method for producing from the target to be imaged a real two-dimensional image filtered through a partly negative filtering spectrum. The negative filtering spectrum can naturally not be carried out by means of a physical filter element alone as the transmittance of the light through a filter is always between 0 and 100%. The possibility to use a negative filtering spectrum would, however, present a possibility to receive from the imaged target information that it is not possible to receive by means of traditional methods.

### SUMMARY OF THE INVENTION

The purpose of the invention is to produce a new method and apparatus for imaging a target so that the final image corresponds to a situation in which it is imaged through a theoretical, at least partly negative filtering spectrum. An especial purpose of the invention is to provide an effective measurement method not limited to the used lighting power or type of light source, whereby practical two-dimensional imaging can be carried out with short exposure times and in a number of practical applications.

An important purpose of the invention is to produce a method and apparatus suitable for imaging ocular fundus that could be used in, for example, diabetes screening.

The invention is based on the idea of producing the filtering spectrum comprising negative parts by photographing the target, typically through a plurality of various filter elements or an adjustable filter, and post-processing the information contained by the various pictures. Thus, in the method of the invention the desired filtering function is not carried out as such directly in the lighting of the target or the picture sensor but the method comprises forming as an end result of accurate, controlled lighting and post processing an image corresponding to a situation in which the lighting of the target has been carried out by means of a desired filtering spectrum comprising negative parts.

One feature of the invention is carrying out the lighting and light detection of the target to be studied so that an effect corresponding to the pre-defined filtering spectrum can be achieved mathematically by using a partial, band-specific image information. The invention especially produces a method in which the exposure adjustment of the target and the imaging are performed band-specifically with each separate used wavelength band according to a pre-determined exposure time, i.e. band-specific partial images with accurately controlled exposure are produced. Typically the imaging is carried out so that each positive and negative part of the filtering spectrum contains many, i.e. at least two, adjacent imaging wavelength bands.

In the method according to the invention a plurality of combinations of exposure times and wavelength channels are determined so that at least a part of the wavelength channels are on the positive area of the filtering spectrum, in which the exposure is determined to essentially correspond with the filtering spectrum and at least a part of the wavelength channels are in the negative area of the filtering spectrum, in which the exposure is essentially determined to correspond with the inverted filtering spectrum. Subsequent to this, a plurality of partial images are made of the target using the said combinations of exposure times and wavelength channels by illuminating the target with the light source, by filtering the light emitted by the source of light and by detecting the light reflected by the target or passed through it by means of an image detector by using a pre-determined exposure time separately for each wavelength channel. Finally, the said image filtered with a partially negative filtering spectrum is formed mathematically of the partial images.

The wavelength channels are preferably chosen by filtering before the target, whereby only the necessary amount of light is directed on the target. This increases the safety of the method especially in medical applications and it also allow lighting the target continuously while keeping the total lighting power small. For example, directing the imaging optics onto the target is easier when using continuous lighting, which is very important when imaging the ocular fundus, as will be described later in more detail.

According to an especially preferable embodiment the exposure time is chosen after the target, i.e. on the detector side, by means of the image detector. A mechanical shutter or electronic control of the detector can be used. This solution maximizes the signal to noise ratio of the imaging and it is especially preferable when used together with light filtering prior to the target and it is compatible with continuous lighting of the target as well.

As the resultant image is mathematically formed, the partial images must be separately saved. This point is the difference between the present method and the methods utilizing essentially integrating measurement and it makes it possible to use partial images for making a plurality of different filtering spectra. Thus the present method is much more flexible than integrating methods.

According to one embodiment an image is formed of the target by means of a plurality of partial images taken at different wavelength channels for forming an image filtered by means a partially negative filtering spectrum so that
a plurality of partial images are taken of the target using the said combinations of exposure times and wavelength channels by lighting the target with the said wavelength channels and by separately choosing an exposure time for each wavelength channel by means of the image detector;
a linear combination of positive images is formed by the partial images of the wavelength channels corresponding with the positive values and a linear combination of negative images from the partial images corresponding with the negative values, and
the linear combination of the negative partial images is subtracted from the linear combination of positive partial images for forming the image filtered by means of the said partially negative filtering spectrum.

The corresponding apparatus comprises
a light source for lighting the target,
a camera for measuring the light either reflected by the target or having passed through the target with a selected exposure time for forming an image of the target,
means for selecting a wavelength channel of the light incident at the camera from a plurality of wavelength channels,
memory means for saving the desired filtering spectrum,
means for defining a plurality of exposure combinations formed by exposure times and wavelength channels by means of a filtering spectrum and wavelength channels so that at least a part of the wavelength channels are on the positive area of the filtering spectrum, on which area the exposure is essentially defined so as to correspond with the filtering spectrum, and at least a part of the wavelength channels are on the negative area of the filtering spectrum, on which area the exposure is essentially defined to correspond with the inverted filtering spectrum,
   wherein the apparatus is arranged to form a linear combination of positive images from the partial images of the wavelength channels corresponding with the positive values and a linear combination of negative images from the partial images corresponding with the negative values and to subtract the negative partial images from the linear combination of positive partial images for forming the image filtered by means of the said partially negative filtering spectrum.

The present invention offers numerous advantages. With the present invention, it is especially possible to use effective and even continuous wide-spectrum light sources in imaging and thus to use short exposure times. Therefore the invention is useful for two-dimensional spectral imaging of real targets and for recognizing small colour differences in these targets. As the exposure and therefore the forming of partial images is made separately for each wavelength band, the image detector, such as a CCD camera or the like, is not saturated and its full dynamic range can be used. Further, separate, wavelength specific images can be separately saved and they can be used as such for studying the spectral properties of the target or should it be desired of the target. In this respect the exposure solution of the invention greatly differs from e.g. the exposure solution described by Fauch et al based on an integrating detector and scanning of light source over the desired wavelength area. Thus the invention is better suited to for high spectral definition two-dimensional imaging applications.

The invention can be used in all applications utilizing digital photography. The invention can especially be utilized in applications in which the lighting of the target by means of certain kind of filtering can yield information that would otherwise be difficult to extract. Such applications exist in e.g. the field of medical imaging. One practical example is imaging the ocular fundus for seeing the damage caused to the retina by diabetes. Such applications require both a very high definition on the image plane and very exact colour separation, both of which can be achieved by means of the method and apparatus according to the present invention. As the method and apparatus are not limited to genuinely positive filtering spectra only, more information can be received from the target than with traditional methods. For example, points of the image plane having the same RGB colour space values can be distinguished because the suitably selected partially negative filter causes different pixel values to the pixels corresponding with them in the final image.

So, in practice the invention allows visualization of mathematical negative filtering spectra and it thus increases the possibilities of spectral imaging. The invention can be used, for example, spectral imaging of the human ocular fundus, in which using the method allows carrying out the effect of partially negative filters available from e.g. principal component analysis by adjusting the spectral form of the image, i.e. it is possible to take main component images or images filtered by other means from the ocular fundus. In the principal component method a plurality of specific vectors of the spectrum space are selected, as a combination of which it is in principle possible to form all possible light spectra, i.e. all colours. This is necessary, as traditional RGB imaging cannot resolve all existing colours. Usually, a spectral image containing a plurality of spectrum channels would have to be taken of the ocular fundus and then calculate the main component spectra as well as the projections of the measured spectra for these main component axes. This can be numerically heavy. By means of the invention the main component picture can be directly formed and subsequent to taking the images it is only necessary to subtract the image corresponding with the negative filter from the positive one and to scale the values of the pixels to the desired value range.

The invention has corresponding applications in the medical fields in imaging tissues, such as skin, as well as other medical spectral analyses. Other applications of the invention include e.g. security applications, such as person identification, analyzing markings containing colour information difficult to forge and identifying forged products, quality control applications as well as spectrum research and optical measurement technology generally in various fields of science, such as space research.

One of the advantages of the invention in comparison with traditional spectral imaging is that by correctly selecting the filtering spectrum the interesting points can be immediately made visually distinguishable. The filtering spectrum can be chosen to pick and separate certain metameric colours, i.e. colours that look the same but are formed by different spectra. When the properties of the filtering spectra are known the presence and location or absence of such in the image can be easily seen in the image.

According to a preferred embodiment the present invention for filtering the lighting of the target according a predetermined filtering spectrum for achieving a similar effect in the imaging of the target comprises the following steps:
taking digital photographs of the target by filtering the lighting of the target with interference filters using, for each photograph, a combination of transmission filter and exposure time determined by the filtering spectrum so that the transmission spectra and exposure times are adapted to correspond with the filtering spectrum on the wavelength bands corresponding to the positive values of the filtering spectrum and to correspond with the inverted filtering spectrum in the wavelength bands corresponding with the negative values of the filtering spectrum,
forming a linear combination of positive images from the images of the wavelength channels corresponding with the positive values and a linear combination of negative images from the images corresponding with the negative values,
subtracting the linear combination of the negative images from the linear combination of positive images, and
scaling the values of the pixels in the final image to a range suitable for visualizing. The relative values of pixels remain unchanged.

As the transmission spectrum of the interference filter cannot usually be adjusted, the adapting is in this embodiment in practice carried out by adjusting the exposure time. Lengthening the exposure time naturally increases the effective transmittance of light in the transmission band of each interference filter and vice versa. On the other hand, if spectrally adjustable filters or light sources are used, it is possible to effect the used wavelength bands in addition to the exposure times.

By a partially negative filtering spectrum we mean a filtering spectrum containing at least one wavelength band comprising positive filtering values and at least one wavelength band comprising negative filtering values. There can also be a plurality of positive and/or negative bands.

In addition to the described apparatus assembly the invention also relates to the components of the apparatus, such as exposure calculation/control unit and/or the imaging unit or a combination of these and a light source, filter arrangement and/or image detector. By means of these components, i.e. modules, the existing spectrum imaging apparatuses can be updated so as to be in accordance with the invention.

The various embodiments of the invention and additional information are described in the following with reference to the appended drawings.

### SHORT DESCRIPTION OF DRAWINGS

FIG. 1 shows an exemplary filtering spectrum, the positive part of the filtering spectrum, the negative part of the filtering spectrum as well as the negative part of the filtering spectrum inverted.

FIG. 2 is an exemplary spectrum view of the wavelength channels possible with 30 narrow-band interference filters.

FIG. 3 shows the apparatus assembly according to a preferred embodiment of the present invention.

### DETAILED DESCRIPTION OF THE EMBODIMENTS

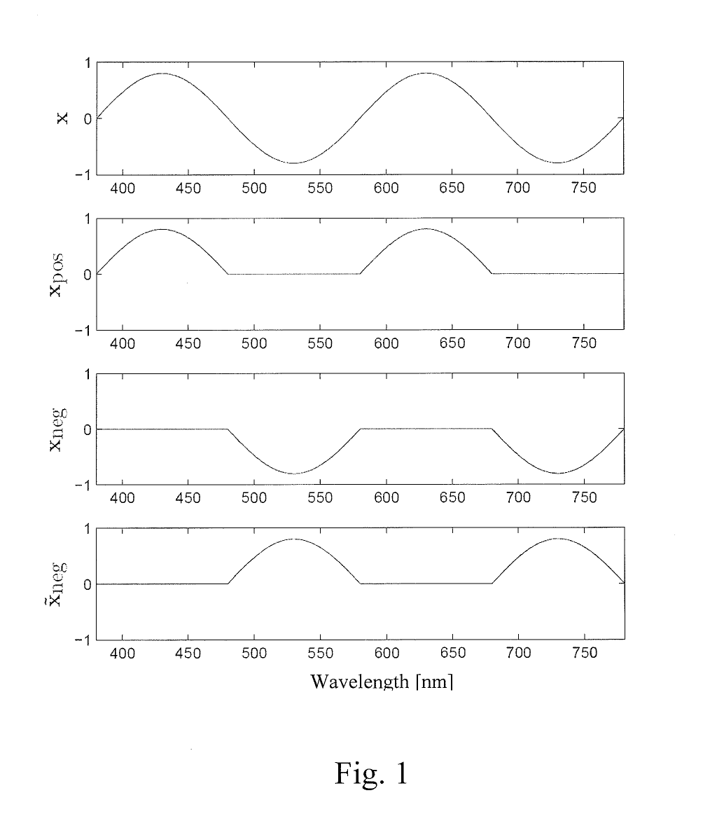

FIG. 1 shows a predefined filtering spectrum x, the positive part xₚₒₛ thereof, the transmittance, according to which the combinations of the wavelength bands and negative part x_{neg} thereof as well as the inversion of the negative parts x^{˜}_{neg} of the exposure times are selected in the negative part of the wavelength bands of the spectrum. In the wavelength bands corresponding with the positive values the combination is directly arranged to correspond with the filtering spectrum.

According to a preferred embodiment the target is illuminated with a wide-band light source, that can, due to the exposure made by means of the used image detector, be e.g. of continuous type. The total lighting power of the light source in the used light range is preferably at least 500 lm, typically about 1000-4500 lm. Even light sources of 10000 lm can be used. The light source can be, for example, an incandescent light source, electroluminescence light source or a gas discharge light source. It is possible to use, for example, halogen light.

The amount of the used wavelength channels is at least 10 in order to be able to carry out the filtering function in practice with a meaningful resolution. In imaging application requiring high spectral resolution, such as imaging of the ocular fundus, up to 25 or even more wavelength channels can be used. Thus, the average lighting power per channel can be, for example, 10-1000 lm, typically 20-150 lm.

In connection with the imaging of the ocular fundus it should be noted that by using narrow-band filters the lighting power/wavelength can be maximized, and thus the exposure times can be minimized, as the light power reception capability of the person is the limiting factor. Thus, in the imaging of ocular fundus at least 25 separately exposed wavelength bands are preferably used.

As has been noted in the above, according to a preferred embodiment of the invention the wavelength channels are selected by means of a plurality of constant filters, such as an interference filter. Interference filters allow forming very narrow wavelength bands, the half value width of which is typically from 5 to 10 nm. The number of used filters is high enough to cover the whole filtering spectrum desired. Typically the imaging, is done on the whole range of visible light, i.e. at a range from 400 to 700 nm. It should, however, be noted that the principle of the invention can also be applied to, for example, the near infrared range (NIR range). The advantage of interference filters is that they can be used with very high-power light sources and thus reduce the measuring time and/or improve image quality.

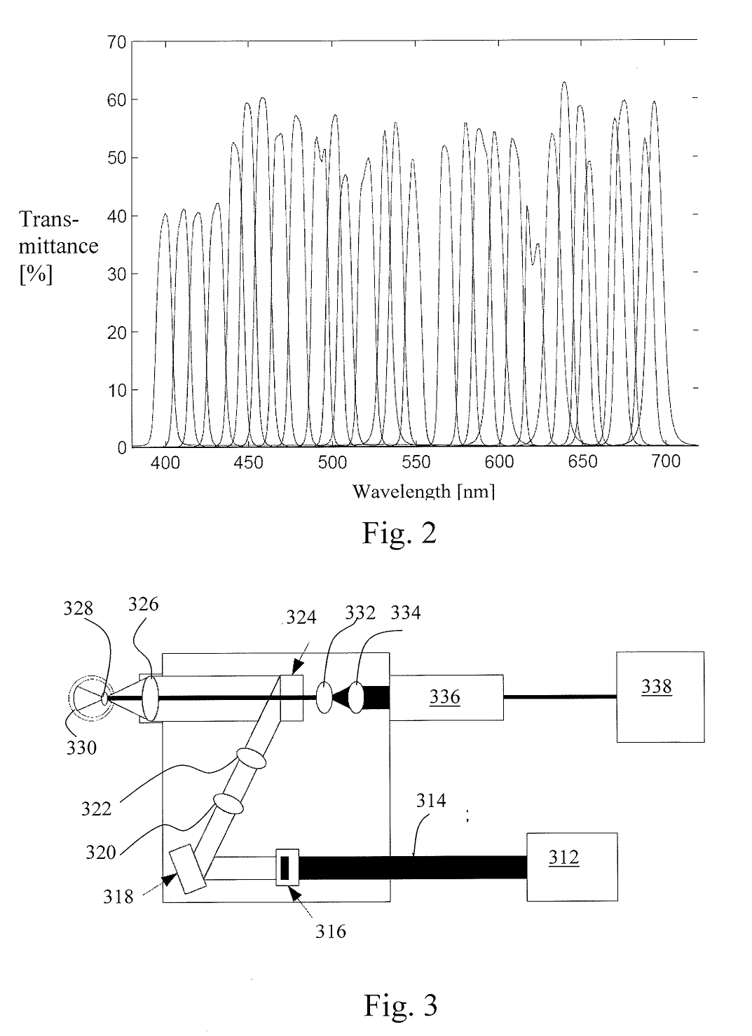

FIG. 2 shows the transmission spectra of 30 interference filters. This kind of filter selection allows imaging in practice on the whole range of visible light.

In an alternative embodiment the used wavelength channels are selected by means of an adjustable optical filter, such as an adjustable liquid crystal filter (LCTF), an adjustable acousto-optical filter (AOTF) or an adjustable monochromator. The adjustable filter is controlled, for example, in steps to produce the whole desired spectrum range in even steps. If desired, the control can be made more frequent in ranges where the best possible spectral definition is needed and/or on the basis of the properties of the used filtering spectrum. The possible light power range of an adjustable liquid crystal filter is relatively high, so it can be used with high-power light sources as well. A monochromator, on the other hand, is only suitable for fairly low light power imaging.

Further, in an alternative embodiment a plurality of light sources are used, by means of which the wavelength bands are produced either directly or further by filtering in order to produce a multi-chromatic light source suitable for the light source. Combinations of the above-mentioned arrangements are also possible.

In a typical application the used wavelength channels cover at least 90% of the filtering spectrum range, typically of the whole range of visible light, and most preferably the whole range of visible light, when measuring the half value widths of the intensities of the bands.

FIG. 3 shows an apparatus according to one embodiment of the invention. The apparatus comprises a light source 312 connected by an optical fiber 314 to a filter unit 316, in which the desired wavelength band is pass filtered. The filtered light is guided via first mirror 318 and optics 320, 322 to the second mirror 324. The second mirror 324 is arranged to reflect the incident light to the target to be imaged, in this case the ocular fundus 330 through a lens 326. In the imaging of ocular fundus the light is directed through the same optics 328, 326 back to the second mirror 324 arranged to allow the reflected light through the necessary optics 332, 334 to the camera 336. In the example of the figure the second mirror 324 comprises a central opening through which the reflected light passes (the diameter of the reflected light is considerably smaller than that of the lighting). From the camera 326 the image information is passed via a communications line to a computer 338 for further processing. One skilled in the art knows that a corresponding optical arrangement can also be carried out in other ways.

The filter unit 316 comprises the necessary means for producing a plurality of narrow wavelength bands, typically a plurality of constant filters or an adjustable filter. The constant filters can be placed in a filter rail or disc, for example, that can be moved or revolved in the filter unit 316 so that at each time one filter is placed on the optical axis of the incident light. For this the filter unit 316 can contain a motor or the like means allowing automatic filter changes. A manual filter change is, however, also possible.

The operation of the imaging apparatus is controlled by means of a control unit, most typically a computer 338 used for collecting and processing data.

The light filter or filters can be located either between the light source and the target or the target and the camera. However, especially in medical imaging and in imaging of other light-sensitive targets it is preferable to filter before the target. This allows minimizing the amount of light power incident on the target at a time and, in case of e.g. imaging the ocular fundus, to minimize the blinding of the target. Further, the amount of dispersed light possibly transferred to the camera can also be reduced.

According to one embodiment the desired spectral form of the filter is achieved by adjusting the exposure time of the camera separately for each wavelength band. This is preferred, as this allows the method to be carried out by short exposure times while using a high-power light source, the minimum switching time of which in itself would be longer than the exposure time. It is even possible to use a continuous light source. The exposure time can be adjusted, for example, by means of the electrical control of camera, the mechanical shutter of the camera or by means of a separate light breaker. Alternatively, a flash-type light can be used and the exposure can be adjusted according to the flash power and/or switching time while the light is directed to the camera and the camera is on during the whole flash. Calculation of exposure times is described in closer detail hereinafter.

In practice, the intensity, camera sensitivity or the filters' transmittance distributions are not ideally smooth, whereby the method typically requires, in addition to the filtering spectrum to be carried out, also detailed information about the used light source, (interference) filters and the imaging device.

The values of the pixels of the final image can be outside the gamut of the used bit space, whereby the image usually typically has to be scaled to a range suitable for visualization. The suitable scaling is preferably made for the final combination image, but a corresponding effect can also be achieved by scaling the values of the partial images. Subsequent to this the image can be visualized by means of a display device, which is of specific importance in the applications of, e.g. medical imaging.

Fundus imaging is one of the most important applications of the invention. Fundus imaging has been established as one of the basic methods of ophthalmologic research, especially because a clear connection has been established between diabetes and structural changes of the ocular fundus. These changes can be found out by means of an ophthalmic fundus camera/fundus camera system (FCS), and the apparatus according to the present invention can also be realized as such. The changes of the fundus, i.e. the retina, can be very small and in order to exactly visualize them already in the initial stage of the changes the spectral definition must be better than in traditional RGB cameras. The partially negative filtering of the present invention brings an improvement, because by suitably choosing the filtration spectrum only the desired, small changes can be brought out in the filtered images.

Fundus imaging is carried out one eye at a time while the patient is looking at a certain point. As the final image is formed from a combination of partial images, the effect of involuntary eye movements must be eliminated as much as possible. If necessary, the imaging at each wavelength band can be made numerous times, whereby it is possible to manually or automatically select the most suitable exposure for imaging. The pupil can be medically dilated prior to the imaging.

A suitable light source for fundus imaging is, for example, a fiber-optical light source provided with at least a 100 W halogen lamp or other white light lamp having a similar lighting power. Such apparatuses are offered by, for example, Schott North America and Osram Corp.

A suitable detector for fundus imaging is a semiconductor-based grayscale camera, the planar definition of which is at least 1000×1000 pixels, preferably at least 2000×2000 pixels. Grayscale camera QImaging Retiga-4000RV can be mentioned as an example.

The above-described means can, of course, used in other applications as well.

According to one preferable embodiment the image filtered by means of a partially negative filter formed by means of the invention is saved in a database that can further be for analyzing the images taken from a similar target. Forming a spectral database of ocular fundus images is mentioned as an especially preferred application. According to another embodiment the image filtered by means of a partially negative filter formed by means of the invention is compared to an image or images in such a database. In the comparison, automatic shape recognition can be used.

In practice the invention can be carried out by defining exposure times on the basis of the following description.

Let m be the dimension of the spectrum space vector and n be the amount of interference filters. In this case the approximation for the desired filter x is calculated in the following equation

SHFt=x, (1)

in which m×m diagonal matrix S contains the radiation spectrum of the light source, m×m diagonal matrix H contains the spectral sensitivity distribution (quantum efficiency) of the imaging device, columns of the m×m matrix F include the transmission spectra of the narrow-band interference filters, the n vector t contains the exposure times for various filters and the m vector x is the transmission spectrum of the desired filter. In case x contains both positive and negative parts, it is divided in two parts: x=xₚₒₛ+x_{neg}, in which the components xᵢ, i=1 . . . m of x are distributed as follows:

$${( {x}_{pos} )}_{i} = \{ \begin{matrix}{x}_{i} , & when {x}_{i} \geq 0 \\ 0 , & otherwise\end{matrix} \\ {( {x}_{neg} )}_{i} = \{ \begin{matrix}{x}_{i} , & when {x}_{i} < 0 \\ 0 , & otherwise\end{matrix} \qquad ( 2 )$$

The positive part and the inversion x^{˜}_{neg}=−x_{neg} of the negative part can be implemented. When marking X=SHF, the relative exposure times tⱼ, j={pos, neg}—for a certain light source, imaging device and filters—of the filters xₚₒₛ and x^{˜}_{neg} are calculated as follows:

tⱼ=(X^{T}X)⁻¹X^{T}xⱼ, (3)

in which xⱼ={xₚₒₛ, x^{˜}_{neg}}. The resulting exposure times are relative, so it is advantageous to norm the vectors tⱼ so that the value of the longest exposure time is 1:

$${\hat{t}}_{j} = \frac{{t}_{j}}{{max}_{i} \{ {t}_{i} \vert {t}_{i} ∈ \{ {t}_{pos} ⋃ {t}_{neg} \} \}} . \qquad ( 4 )$$

When the target is imaged, the actual exposure time τ of the interference filter corresponding with the longest exposure time τ by imaging the target with light illuminated through the said filter. Thereby the final exposure times used in imaging are

(tⱼ)_{final}=τ\hat{t}ⱼ. (5)

The actual imaging is made separately by using the corresponding exposure times calculated for each interference filter. This results in n digital images: for the filter xₚₒₛp (0≦p≦n) images Iₚₒₛ and n-p images I_{neg} for the filter x^{˜}_{neg}. A digital image I simulating the effect of the desired filter xₚₒₛ+x_{neg}=xₚₒₛ−x˜_{neg} can be calculated as the linear combination of the saved images:

$$I = {\sum }_{i} {( {I}_{pos} )}_{i} - {\sum }_{j} {( {I}_{neg} )}_{j} . \qquad ( 6 )$$

Part of the values of the pixels of the image I are probably below the normal value range [0, 2b^{I}−1], in which b is a bit rate. Due to this the values of image I are scaled on the correct range as follows:

$${I}_{1} = I - min \{ p \vert p ∈ I \} , \\ {I}_{final} = \frac{{2}^{b} - 1}{max \{ q \vert q ∈ {I}_{1} \}} {I}_{1} , \qquad ( 7 )$$

in which I_{final} is the final digital photograph performing the function of the filter x. The values from equation 7 can, of course, finally be rounded to integers.

The embodiments, examples and the appended drawings described above are meant to serve as examples of the practical implementations and advantages of the invention and they are not to be construed as limiting the invention. The invention is described in the appended patent claims that are to be interpreted in their full extent, with regard to equivalent solutions.

1. A method for forming an image of ocular fundus, wherein said image is filtered by means of a partially negative filter spectrum using a plurality of partial images taken at a plurality of wavelength channels, the method comprising
determining a plurality of combinations of exposure times and wavelength channels so that at least a part of the wavelength channels are in the positive area of the filtering spectrum, in which the exposure is determined to essentially correspond with the filtering spectrum, and at least a part of the wavelength channels are in the negative area of the filtering spectrum, in which the exposure is determined to essentially match the inverted filtering spectrum,
taking a plurality of partial images of the ocular fundus by lighting the ocular fundus with a light source, by filtering the light emitted by the light source and by detecting the light reflected or transmitted by the ocular fundus by means of an image detector,
saving the partial images, and
forming, by calculation, the said image filtered with the partially negative filtering spectrum of the partial images,
the method further comprising
taking the partial images separately for each wavelength channel by using an exposure time determined for said wavelength channel and selected by means of the image detector.

2. A method according to claim 1, wherein the filtered image is formed by
forming a linear combination of positive images from the partial images of the wavelength channels corresponding with the positive values and a linear combination of negative images from the partial images corresponding with the negative values, and
subtracting the linear combination of the negative partial images from the linear combination of positive partial images.

3. A method according to claim 1, comprising scaling the values of the pixels of the final image to a range suitable for visualization and preferably showing the filtered image on a display screen.

4. A method according to claim 1, wherein the number of wavelength channels is at least 10, preferably at least 25.

5. A method according to claim 1, wherein the ocular fundus is illuminated with a wide-band light source, preferably by means of a continuous-type light source, the total lighting power of the light source being at least 500 lm, preferably 1000-4500 lm.

6. A method according to claim 5, wherein the wavelength channels are selected by filtering before the target.

7. A method according to claim 1, wherein the wavelength channels are selected by means of a plurality of constant filters, such as interference filters.

8. A method according to claim 1, wherein the used wavelength channels are selected by means of an adjustable optical filter.

9. A method according to claim 1, wherein the exposure times are selected using a mechanical shutter of the image detector or using an electronic control of the image detector.

10. A method according to claim 1, wherein information about the spectral intensity distribution of the used light source and/or the sensitivity distribution of the used image detector are as well used for determining the exposure combinations.

11. A method according to claim 1, wherein the used wavelength channels cover at least 90% of the total range of visible light, preferably the whole range of visible light, when measuring the half value width of the intensity.

12. A method according to claim 1, wherein the exposure times tⱼ are determined according to equation
tⱼ=(X^{T}X)⁻¹X^{T}xⱼ, (3)
in which X is a matrix taking into account the radiation spectrum of the light source, the sensitivity distribution of the light detector and the used wavelength channels and xⱼ is a filtering spectrum vector containing a partially uninverted filtering spectrum and a partially inverted filtering spectrum.

13. A method according to claim 1, wherein the light is directed to the ocular fundus through a partially transparent mirror and further to the image detector through the said partially transparent mirror.

14. (canceled)

15. (canceled)

16. An apparatus for forming an image of ocular fundus by means of a plurality of partial images taken at different wavelength channels for forming an image filtered by means a partially negative filtering spectrum, the apparatus comprising
a light source,
a lens for directed light from the light source to the ocular fundus for lighting the ocular fundus,
a camera for measuring the light either reflected or transmitted by the ocular fundus with a selected exposure time for forming an image of the ocular fundus,
means for selecting a wavelength channel of the light incident at the camera from a plurality of wavelength channels,
memory means for saving the desired filtering spectrum,
means for defining a plurality of exposure combinations formed by exposure times and wavelength channels by means of the filtering spectrum and the wavelength channels so that at least a part of the wavelength channels are on a positive area of the filtering spectrum, on which area the exposure is essentially defined so as to correspond with the filtering spectrum, and at least a part of the wavelength channels are on the negative area of the filtering spectrum, on which area the exposure is essentially defined to correspond with the inverted filtering spectrum,
wherein that the apparatus is arranged to select a predetermined exposure time with the camera and to form and to save the partial image separately for each wavelength channel.

17. An apparatus according to claim 16, being adapted to form a linear combination of positive images using the partial images corresponding with wavelength channels on the positive areas of the filtering spectrum and a linear combination of negative images using the partial images corresponding with the negative areas of the filtering spectrum and to subtract the linear combination of the negative images from the linear combination of positive images for forming an image filtered by means of a partially negative filtering spectrum.

18. An apparatus according to claim 16, wherein
the light source is a wide-band light source,
the means for selecting the wavelength channel comprise means for forming at least 10 different wavelength channels for covering at least 90% of the range of visible light when measuring the half-value width of the intensity, preferably a plurality of fixed filters, such as interference filters.

19. An apparatus according to 16, wherein said means for selecting the wavelength channel are located between the light source and the ocular fundus.

20-22. (canceled)
